(19) United States
(12) Patent Application Publication
(10) Pub. No.: US 2018/0294059 A1
(43) Pub. Date: Oct. 11, 2018

(54) Mental Health Modeling Language

(71) Applicant: Robert Lopez, (US)

(72) Inventors: Vidushi Savant, Los Altos, CA (US); Robert Lopez, San Mateo, CA (US); Vikas Kedia, Los Altos, CA (US)

(73) Assignee: Savant Care Inc., Los Altos, CA (US)

(21) Appl. No.: 15/928,088

(22) Filed: Mar. 22, 2018

Related U.S. Application Data
(60) Provisional application No. 62/475,123, filed on Mar. 22, 2017.

(51) Int. Cl. G16H 50/20; G16H 10/60; G06N 3/08

(52) U.S. Cl. CPC G16H 50/20; G06N 3/08; G16H 10/60

(57) ABSTRACT

The invention is a novel, internet-enabled doctor-patient workflow system comprising, inter alia, an “intelligent” electronic health record and healthcare management process, offering an interactive “machine-learning” electronic health record and medical management system. The invention features inputs and commands from doctors through the use of a conversation pane (conversation window). The invention uses artificial intelligence and machine learning algorithms to accomplish routine activities via short code commands and auto-fill menu-populating technology which adapts itself to a particular physician's style as the System is used.

### FIELD OF TECHNOLOGY

The present invention relates to Healthcare Technology, and more specifically to technology-based tracking, recognition, memorialization, categorization and filing of interactions between providers and patients during medical appointments.

### BACKGROUND AND DESCRIPTION OF RELATED ART

In current sessions between a provider and a patient, the provider (user) cannot maintain eye contact with the patient while taking appropriate notes and writing of facts for documentation of the encounter. Providers are required to document thorough information regarding the patient such as their vitals, chief complaint, mental status exam, review of systems, and SOAP (Subjective, Objective, Assessment, and Plan) notes.

Current data entry into electronic health records or paper writing of notes requires the doctor to break eye contact and focus on their data entry tool (be it digital, or hard copy) to record the appropriate data into the correct fields of the entry. Patients complain that the doctor ‘is not paying attention’ to them, or ‘spends more time and attention on the computer than me.’

Related Arts that have attempted to resolve this issue are:

1. US PAT. APPLICATION 20140222462A1: SYSTEM AND METHOD FOR AUGMENTING HEALTHCARE PROVIDER PERFORMANCE, by inventor Ian Shakil and Pelu Tran. This invention utilized a recording device to record data during doctor-patient interactions. Problems with this design are HIPAA compliance risks and the need for the provider or third party to review the recorded data to make the appropriate entries into the patients' records.

2. US PATENT APPLICATION 20050055246A1: PATIENT WORKFLOW PROCESS, Inventor Jeffrey Simon. This invention Claims a method for optimizing the patient workflow process addressing the healthcare professional-patient encounter utilizing an electronic system interfacing with an electronic medical record application. This invention further utilizes a tablet PC to allow the user (provider) to enter data via the tablet PC using a series of check boxes and drop-down menus.

Problems with this design are the overall complexity of the design which requires the user to be focused on which boxes they're clicking, searching for the correct data fields, and/or ensuring they're selecting the correct drop down items. The problems still persist with the provider-patient relationship straining due to attention to the computer coming before attention to the patient.

3. U.S. Pat. No. 7,936,925B2, PAPER INTERFACE TO AN ELECTRONIC RECORD SYSTEM, Inventors Nathaniel Martin, Naveen Sharma et al. This invention utilizes a physical object (paper) to require the user (provider) to write data on the paper in bulk, make drawings, and/or select/shade in different boxes on the form. Upon completion, the form is scanned by a computer system which translates the checkboxes into answers and attempts to transcribe or record any images or drawings or handwritten notes.

Problems with this design are (again) the overall complexity of the form which requires the user to constantly look down to check appropriate boxes and/or requires the unit to focus on their written work in the correct fields for entry. Provider-patient relationship is still strained due to the need to focus on the object being used for compliance and compatibility with the scanning mechanism. What is needed is a System that solves the aforementioned issues.

The herein-disclosed invention executes a novel patient workflow process on any internet enabled a computing device to increase participation of the provider in patient's reporting and conversations and streamlines the visit to allow providers to adequately treat patients in the amount of available time. This creates an increase to practice revenue, enhances data available for reimbursements, and provide an efficiency increase in provider-patient visits, all of which increases both the doctor's and the patient's quality of life. The invention maintains and improves patient-provider communication optimizing the encounter and the documenting procedures normally required by regulation, compliance, or contracting. The invention, as a point-of-care product, is delivered on a computing device through a secure wireless or wired network which interfaces with a secured and protected cloud server. The invention speeds the collection of information while allowing the physician to maintain eye contact with the patient. The invention allows the doctor to effortlessly search, organize and display any information on patients, prescriptions, and symptoms, in real times and juxtapose the data for comparative analysis without the need to break focus and attention from the patient. Using embedded encryption and compaction technologies, the invention assures patient data is secure and meets HIPAA requirements.

The invention improves Doctor/Patient Interaction, communication, and relationship. The invention utilizes inputs and commands from doctors through the use of a conversation pane (conversation window). Routine activities are speeded by short code commands, auto-fill, and suggested text adapted to each physician's style using artificial intelligence and machine learning algorithms. Using the invention will reduce annual patient workflow costs, due to electronic collection and submission of protected health information, transcriptions, coding, prescription, insurance, and referral information. Furthermore, the invention offers clinical recommendations to the provider based on aggregate clinical data collected across all “anonymized” patient data with similar diagnosis, medical profiles, and medication histories. With this insight, a provider can be advised on medications or treatment plans that they may not have been considering or knowledgeable above for ailments their patients are presenting, which will lead to better clinical outcomes.

### SUMMARY

The invention relates to a novel, internet-enabled doctor-patient workflow system comprising, inter alia, an “intelligent” electronic health record and healthcare management process, offering an interactive “machine-learning” electronic health record and medical management system. The invention features inputs and commands from doctors through the use of a conversation pane (conversation window). The invention uses artificial intelligence and machine learning algorithms to accomplish routine activities via short code commands and auto-fill menu-populating technology which adapts itself to a particular physician's style as the System is used.

### BRIEF DESCRIPTION OF THE DRAWINGS

FIG. 1 is a flow diagram of one embodiment of the System;

FIG. 2 is a flow diagram of an alternative embodiment of the System;

FIG. 3 is an exemplary diagram of the Central Skills Manager and Conversation Pane functions of the System;

FIG. 4 is an exemplary diagram of the “event prompt” functionality of the System;

FIG. 5 is an exemplary diagram of the “neural network confirmation” functionality of the System;

FIG. 6 is an exemplary diagram of the “current medications” functionality of the System within the Central Skills Manager;

FIG. 7 is an exemplary diagram of the Deficiencies-Finding functionality of the System;

FIG. 8 is an exemplary diagram of the Help-Desk Shorthand functionality of the System;

FIG. 9 is an exemplary diagram of the Confirmation Notification functionality of the System;

FIG. 10 is an exemplary diagram of the Follow-Up Appointment functionality of the System;

FIG. 11 is an exemplary diagram of the Time-Calendar functionality of the System;

FIG. 12 is an exemplary diagram of the Completion Notification functionality of the System;

FIG. 13 is an exemplary diagram of the “Predictive Behavior Determination and Output Notification” functionality of the System.

### DETAILED DESCRIPTION OF ILLUSTRATIVE EMBODIMENTS

Mental Health Modeling Language (hereinafter “MHML”) embodies the use of neural networks, a skills manager, and user inputs to efficiently and accurately update patient health information through the use of a conversational pane on a computing device. Data entered by the user is evaluated by the system for the type of ‘skill’ being affected and the data points (details, notes, parameters, request types, etc. . . . ) of the skill to automatically appropriate those data points into fields of entry for that particular skill. This allows the user to maintain eye focus with their client (patient) and reduces the time needed to spend searching or clicking through different fields for data entry.

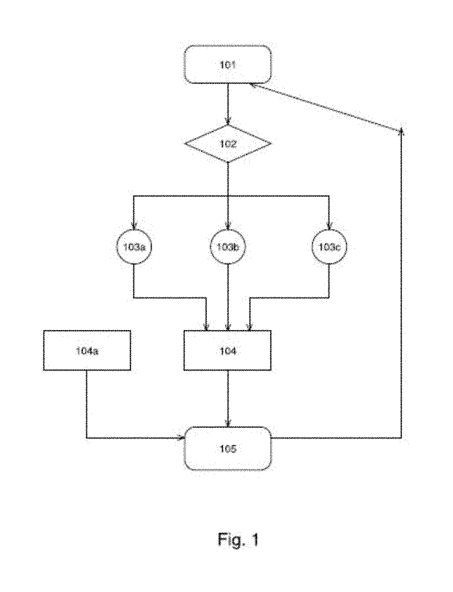

FIG. 1 provides a schematic flow diagram of an embodiment of this process. A Central Skills Manager 101 comprise(s)/access(es)/prompt(s) provider Skills 102, which comprise(s)/access(es)/prompt(s) Medications 103a, Coding 103b and Allergies 103c. This stage then comprise(s)/access(es)/prompt(s) Neural Network Output(s) 104, which comprise(s)/access(es)/prompt(s) a Conversation Pane 105. Simultaneously, Doctor Input 104a contributes to and comprise(s)/access(es)/prompt(s) the Conversation Pane 105. Information from the Conversation Pane 105 is then sent to the Central Skills Manager 101, and so on.

FIG. 1 therefore illustrates a technology architecture diagram of the interaction between a doctor/medical-provider/similar clinical personnel, conversation pane, central skills manager, non-exhaustive list of skills, and neural network for basic data entry. Conversation pane, central skills manager and neural network output collaboratively interact with the doctor's input to provide updates to the user (doctor) and make modifications to the given patient's electronic health records in the appropriate fields of the database in which such data should be entered within.

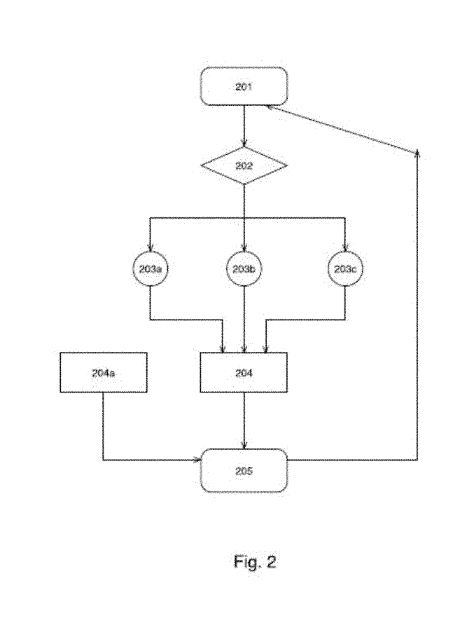

FIG. 2 illustrates a technology architecture-e diagram of the interaction between a user (medical provider, clinical user, etc.) conversation pane, central skills manager, non-exhaustive list of skills, and the neural network for basic data entry while the neural network is enabled to use predictive abilities to interact with the provider.

Specifically, Central Skills Manager 201 comprise(s)/access(es)/prompt(s) provider Skills 202, which comprise(s)/access(es)/prompt(s) Medications 203a, Coding 203b and Allergies 203c. This stage then comprise(s)/access(es)/prompt(s) Neural Network Output(s) 204, which comprise(s)/access(es)/prompt(s) a Conversation Pane 205. Simultaneously, other Doctor/Provider/User Input 204a contributes to and comprise(s)/access(es)/prompt(s) the Conversation Pane 205. Data from the Conversation Pane 205 is then sent to the Central Skills Manager 201, and so on.

Within the conversation pane, central skills manager and neural network output now becomes a two-way interactive loop to interact with the doctor's input to provide clinical recommendations based on data received from the skills database and central skills manager commands to the user (doctor) given the current patient's electronic health records and the appropriate field or subfields of the database in which the clinical relevance has been specified by the user (i.e. diagnosis, suicidal ideation, medications attempted, etc. . . . )

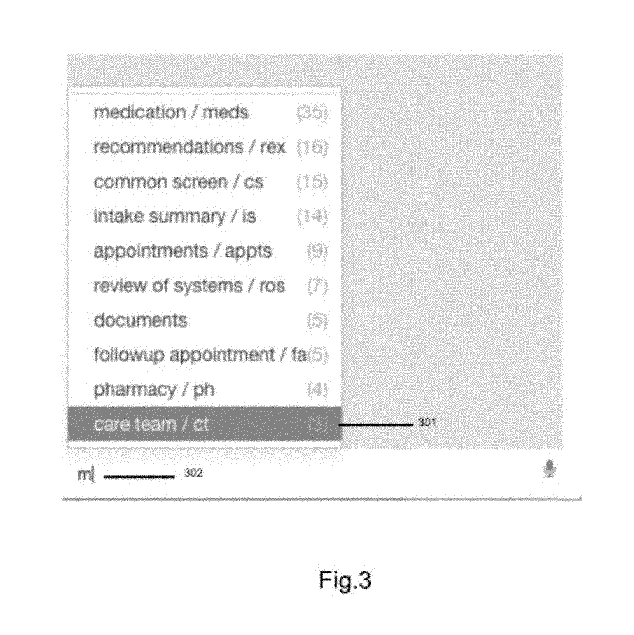

FIG. 3 is a demonstration of the user entering data into the conversation pane. When data is starting to be entered, the user can enter any abbreviations or short code (e.g. “MLE” for “Major Life Events”, “m” for ‘medications”, “dx’ ‘for . . . diagnosis”, etc. . . . ) or full text of ask ill into the conversation pane. Upon entering the data into conversation the pane interacts with the Central Skills Manager which controls different ‘Skills’ the neural network is able to identify and interact with. In this fashion, the user/doctor can choose the Care Team menu option 301, for example, do then type “m” prompt 302 to complete the function with just a one-letter prompt 302.

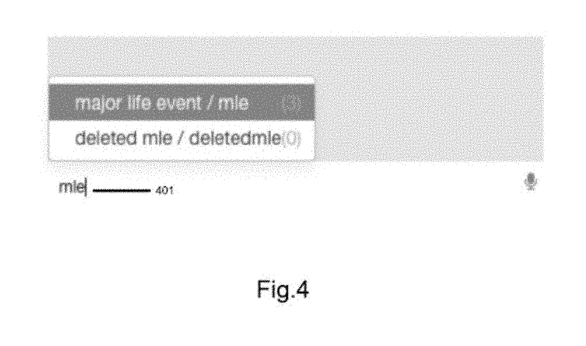

FIG. 4 is a demonstration of the user entering data into the conversation pane for a major life event. After the provider identified the skills (i.e. MLE), the conversation pane prompts the user to enter the “Event” (enter a descriptor or name for such event). In this example, the user entered ‘Married’ and upon doing so, the conversation pane prompts the user to enter the start date in a prompt region 401 below. Start date can be entered in any plain language speech (i.e. “3 years ago. yesterday. Five months ago, etc.” and/or using actual date figures (Mar. 7, 2015; Mar. 7, 2015, etc. . . . )

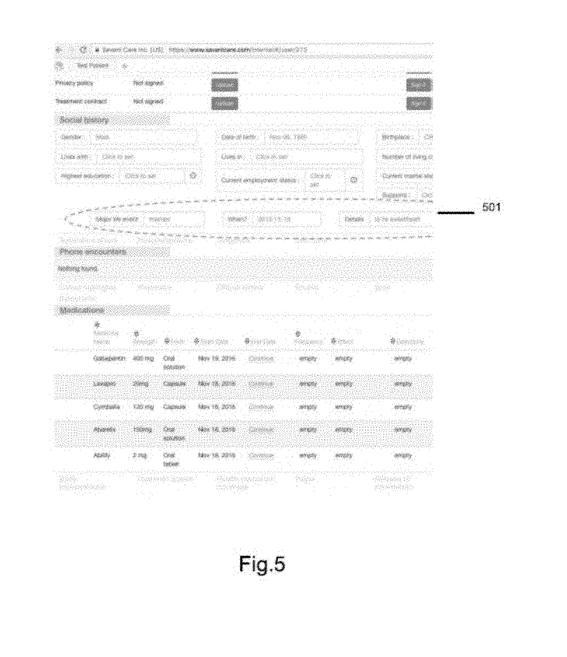

FIG. 5 is a pictorial illustration of a skill being updated through the conversation. The conversation pane has routed the data to the central skills manager, which has identified the correct skill and fields for the data to be updated within. The data is then auto populated (transferred from the conversation pane to the skill and on to the database) and the skill advises the neural network that the data has been successfully updated. The neural network then provides the confirmation 501 (circled in dotted oval for emphasis) via a message to the user that such item has been successfully added and/or updated through the conversation pane.

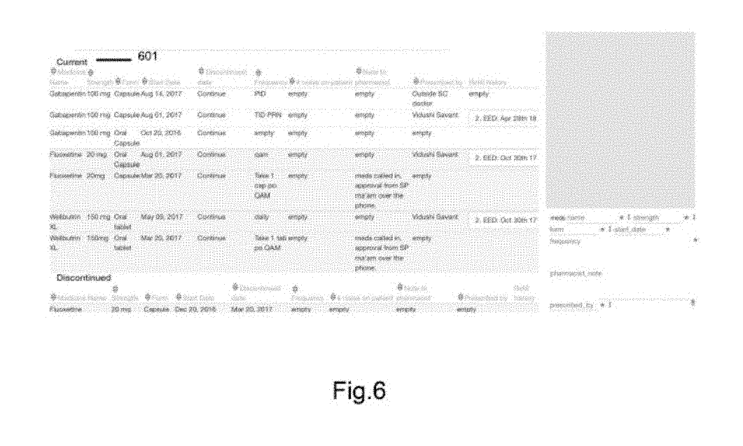

FIG. 6 is a demonstration of a user entering in the meds command through the conversational pane. The conversation pane has routed the ‘meds” command to the central skills manager, which has identified the request as the medication skills and has populated the current medications 601 and discontinued medications on the left for the user, as well as data fields required for entry of new data to inputed into the data base.

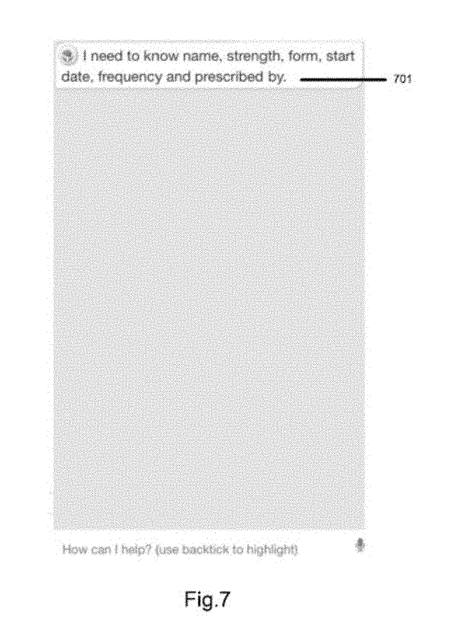

FIG. 7 is a demonstration of a user entering no data after calling upon the medication skill in the conversational pane. The input (or lack thereof) from the user is reviewed by the neural network which has determined that the data is not sufficient for processing a prescription and has provided a common language response to the user of what deficiencies 701 need to be corrected.

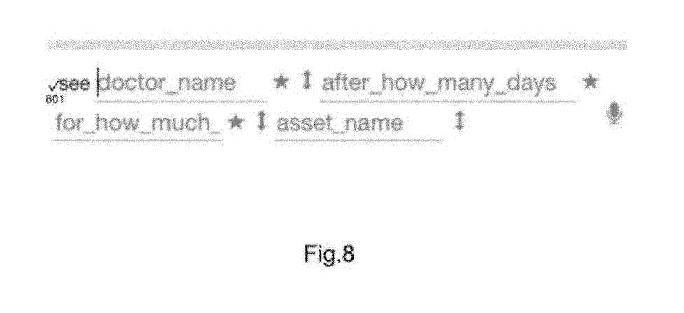

FIG. 8 is a demonstration of a user entering the skill ‘see’ 801 which is shorthand for ‘see mein.’ ‘This skill calls upon the selected provider's calendar after X date or days is specified and provides the patient with a ‘smart’ Help Desk Request where the user can select an available date and time from the provider's future availability. The asset field is to denote any special room or asset the user wishes to allocate (i.e. video conference).

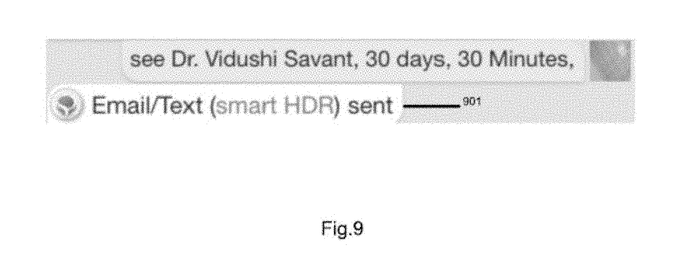

FIG. 9 is a demonstration of the user fully executing the see me in command with the provider Dr. Vidushi Savant for 30 days and 30 min's specified. The Neural Network responds to the user after completing the task, thereby giving a Confirmation Notification 901 that it has sent both an email and an SMS to remind the patient to use the smart Help Desk Request (HDR) to self book the appointment.

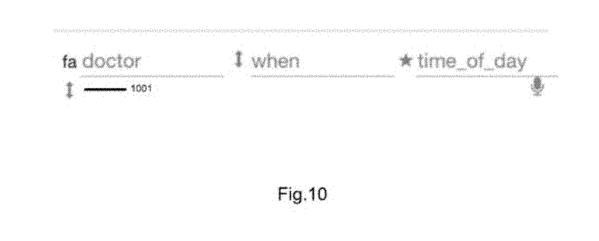

FIG. 10 is a demonstration of the ‘follow up appointment’ skill (hereinafter “FA.”) In FIG. 10, the follow up appointment (hereinafter “fa” 1001) is offered, suggesting the doctor, the place and the time of day for said “fa” 1001 follow-up appointment.

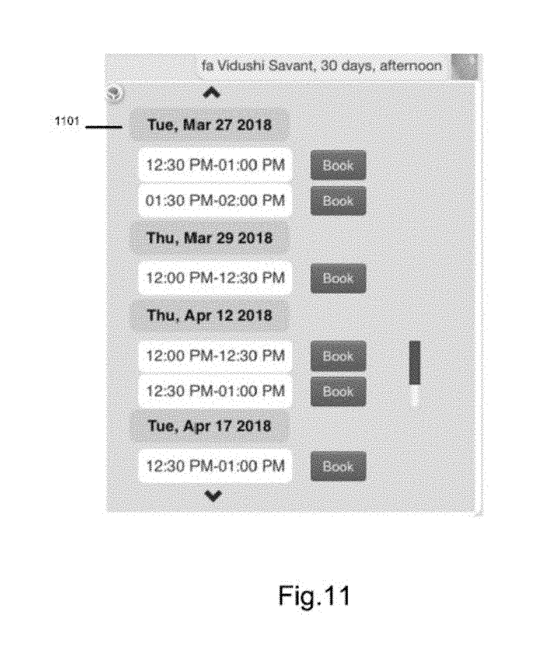

FIG. 11 shows a GUI (herein the time-calendar menu 1101) within the Conversational Pane resulting from an open-ended data entry, created as a secondary-level interaction.

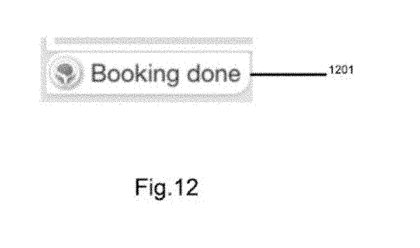

FIG. 12, shows a Completion Notice 1201 from the neural network, indicating that the requested skill “FW1” action was completed.

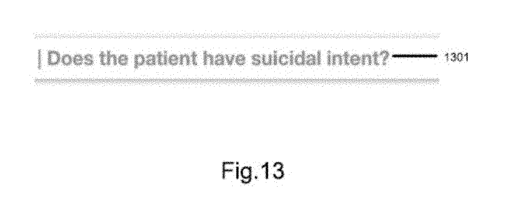

FIG. 13 shows an exemplary output of the System, here an output showing the System's Predictive Behavior, of the neural network. The question “does the Patient have Suicidal Intent?” is a ‘predictive behavior output notification’ 1301 herein formed as a question. Here, the predictive behavior output notification 1301 requests that the provider to extract more information from the patient so the neural network can assist in the clinical evaluation.

### FURTHER DISCLOSURE AND ALTERNATIVE EMBODIMENTS

With MHML embodiment of a neural network between the Doctor Input and Central Skills Manager, this allows machine learning algorithms to understand and recognize patterns and behaviors of a provider and how certain ailments and diagnosis are being treated.

Within the interaction, a secondary language has been designed to allow the user (provider) to enter a short code, abbreviations, or other generally recognized descriptors for the information being requested.

For example, suppose a provider wished to prescribe aripiprazole to their patient. Instead of using the mouse to click into and add data into various fields, a provider can type “medication aripiprazole” or in short “m” or “meds” then “aripiprazole” to input the data into the correct entry fields.

The advantages of this method are:
1. Allows Provider to maintain eye contact with the patient, because they can type without looking at the screen versus using a Graphical User Interface (hereinafter “GUI”) to find the appropriate field box.
2. Reduces the time to input data as the provider can type “m” or “medication” then the name of the medication to add the item to the Electronic Health Record (hereinafter “EHR.”)

In internal tests, it reduced the time to input data from 10 seconds to 2 seconds.

In a 30 minute appointment with a patient, the doctor has to enter an average of 50 data points. Without the system, the user (provider) would spend approximately 10 minutes of the session breaking eye contact and attention from the client (patient) to focus on finding data fields within their electronic records interface to input values, and/or be forced to continue documenting the encounter outside of the allotted appointment time. The 8 minutes and 30 seconds taken is given back to providing attention to the client (patient.)

As a secondary example, let's say a patient said: “I'm allergic to dust.” In the past, the doctor would need to look at their EHR system, find the panel for “allergies” click “add” or some symbol denoting an addition such as a “+” symbol, then input the field value.

With the MHML, the provider need only type “a dust” and the entry is automatically created. After completion of the user input, the data is appropriately stored within the correct fields within the database category for allergies without the need for the user to manually locate and enter the data therein.

The skill being invoked need not be an isolated (single) data point or a closed-end entry. For example, returning to FIG. 9, shows the ‘fa’ being entered into the conversation pane. When fully executed with requested data parameters as demonstrated below in FIG. 9, the conversational pane provides a GUI within the conversational pane that responds to the user's secondary-level interaction. Upon clicking the “book” option, the skills manager recycles the ‘book’ instructions back into the skills function of the ‘fa’ command as an input request to record the data for the given date and time selected, the final output is recorded by the neural network and a response is given as shown in FIG. 10.

### Internal Clinical-Use Testing

For the clinical test, system was deployed the at our private behavioral health medical clinic. Five users from different backgrounds and experience were trained with the same Electronic Health Record systems with 20 hours each via the Mental Health Modeling (MHML) and with a standard Graphic User Interface (GUI).

The following results and findings occurred:
1. It was found that there are exist 2 distinct advantages of MHML over GUI:

1. GUI requires precision where the user is needing to constantly scan the screen to ensure accuracy, whereas MHML requires a glance at the screen.

2. In GUI switching from one panel to another panel is difficult and requires the user to focus versus learned memory commands in MHML where switching from one skill to another is done through key strokes or an input method accepted by the terminal.

Furthermore, if something as seemingly innocuous as color scheme or ordering of panels in the GUI is changed, the user becomes disoriented and can not reorient themselves in a timely manner. Whereas, in a MHML platform, color schemes, re-ordering of the database and skills set, enhancement of the skill set, or modification of the skills themselves, do not affect the user's ability to call upon, interact with, or modify the skills.

What is claimed is:

1. A method of inserting and recalling medical and patient-related data within an existing medical database, Electronic Medical Record, or an Electronic Health Record System, comprising a User and a Controller featuring:
a conversational element that accepts written, spoken, typed, or other stimuli from the user;
a Skills Manager that reads, writes, executes, and interprets said input from the user and call upon a skills database;
said skills database accepting at least one input and generating at least one report output of data points within a protected record, said report output directed to a neural network to relay to said user; and
a Neural Network Manager interface that acts upon input from the user and said database output and converses in natural language
in written prose, spoken frequencies, text, and other stimuli to the user; and
a Conversational pane input field that accepts written and oral methods of communication from the user.

2. The method of claim 1, wherein the application object instantiations form a data manipulation for a specific data point within a medical file.

3. The method of claim 1, of receiving, presenting, and allowing client-side manipulation of the medical data further comprising the steps of:
receiving a transfer of medical data from a server, database, local drive, Hard Drives, storage devices, memory, RAM, ROM, USB drive, or other digital storage mediums;
interpreting the received data so as to generate a secondary data structure, an object oriented environment, and instances acting on the object oriented environment;
generating a presentation of a second portion of the data using the objects; and
allowing manipulation of the presentation through the objects, wherein the interpreting and generating steps are performed by the method herein described.

4. The method of claim 1, of sending data to allow presentation and provider (clinical user)-side manipulation of the data, further comprising the steps of: Transferring the data from a storage location, the first portion of the data comprising of structures and instructors for generating second data structures to form the data necessary to enter with the medical record from either the user or the data to the res and instruction for generating the second data structures from the first data structures, wherein the first portion of the data can be received at the client terminal.

5. The Method of claim 1, of employing machine learning to predict behavior of a provider or user within a clinical or medical setting, said method compromising:
identifying diagnosis history, and
identifying medication history, and
identifying prior treatments recommendations, and
identifying clinical outcomes of recommendation, and
searching the diagnosis and medication history and
other relevant skill sets groups across the server database, wherein
skills of particular interest comprise:
diagnosis, medications, clinical rating scales, review of systems, mental status exams, and/or other pertinent facts the neural network or user may deem medically necessary for evaluation of a particular condition or patient;
said server database comprising digital storage means;
said method further dentifying relationships and correlational patterns between skill sets pertinent to matter being examined;
and aggregating clinical data to calculate the probability of a recommendation being clinically effective for the patient or subject being examined; and
presenting such findings to the user through the conversational pane for the user to review.

6. A method of employing machine learning to predict and recommend data entry to a provider or user within a clinical or medical setting, the method compromising:
Identifying skills and database fields where data appears lacking or missing, incomplete, expired, invalid, or requiring an update and
Providing a recommendation to the provider through the conversational pane or GUI to obtain or require such information to be updated or corrected, and
Provide a secondary interface within such the conversation pane or GUI to enter such data should the provider or user so decide to enter the information.
